(19) United States
(12) Patent Application Publication
(10) Pub. No.: US 2008/0306493 A1
(43) Pub. Date: Dec. 11, 2008

(54) CLIPPING INSTRUMENT FOR AN ENDOSCOPIC SURGICAL DEVICE

(75) Inventors: Hiroaki SHIBATA, Saitama (JP); Tomohiro KAWANO, Tokyo (JP)

Correspondence Address: GREENBLUM & BERNSTEIN, P.L.C., 1950 ROLAND CLARKE PLACE, RESTON, VA, 20191, US

(73) Assignee: HOYA CORPORATION, Tokyo (JP)

(21) Appl. No.: 12/131,290

(22) Filed: Jun. 2, 2008

(30) Foreign Application Priority Data
Jun. 11, 2007 (JP) 2007-153512

(51) Int. Cl. A61B 17/10

(52) U.S. Cl. 606/143; 606/142

(57) ABSTRACT

A clipping instrument for an endoscope is provided. The clipping instrument includes a flexible sheath, an operation wire, at least one detachable clip assembly, including a pair of openable and closable clipping arms and a fastening ring to encircle proximal portions of the clipping arms, arranged in vicinity of a distal end portion of the flexible sheath. The clipping arms can be projected forward and rotated in opening directions to open outside the flexible sheath to self-openable positions, and to be rotated in closing directions to clip onto a treatment object outside the flexible sheath according to forwarding and retracting movements of the fastening ring through the operation wire. The the pair of clipping arms are configured to be respectively rotatable about an axis independently from the movement of the fastening ring by external force and can be further opened extendedly from the self-openable position by the external force.

### BACKGROUND OF THE INVENTION

The present invention relates to a surgical device having a clipping instrument to be inserted through a forceps channel of an endoscope.

Generally, in a clipping instrument for endoscopic surgeries, a clip having a pair of openable/closable arms and a fastening ring loosely fit around proximal portions of the arms is arranged in a distal end portion of a flexible sheath. The clip is configured such that the arms open and close as an operation wire, arranged in parallel with an axial direction of the flexible sheath, is advanced toward the distal end to push the fastening ring and retracted toward a proximal end to pull the fastening ring. The clip can be detached from the operation wire with its arms closed according to the operations to the operation wire. Such a configuration of the clipping instrument is disclosed in Japanese Patent Provisional Publication Nos. 2002-191609 and 2006-87537, for example.

When the clip of such conventional clipping instruments disclosed in the aforementioned publications are operated to be clipped onto in vivo tissue to indwell inside a body cavity, the arms are fully opened to their extent to be pressed onto the tissue and closed.

However, when excessive force is applied from the body tissue to the already fully-opened arms which are pressed onto the body tissue, as shown in FIG. 10, one or both of the arms 91 can be forcibly bended and damaged.

### SUMMARY OF THE INVENTION

In view of the foregoing drawback, the present invention is advantageous in that an endoscopic surgical device with a clipping instrument, of which arms can be extendedly opened further from their self-openable positions by external force without being damaged, is provided.

According to an aspect of the present invention, there is provided a clipping instrument for an endoscope. The clipping instrument includes a flexible sheath to be inserted into an instrument channel of the endoscope, an operation wire, which is configured to be forwarded and retracted in an axial direction of the flexible sheath, and at least one detachable clip assembly, including a pair of openable and closable clipping arms and a fastening ring to encircle proximal portions of the clipping arms. The detachable clip assembly is arranged in vicinity of a distal end portion of the flexible sheath with the clipping arms closed, and the clipping arms are configured to be projected forward and rotated in opening directions to open outside the flexible sheath to self-openable positions, in which the clipping arms are opened by an effect of the fastening ring to their self-openable extent, and to be rotated in closing directions to clip onto a treatment object outside the flexible sheath according to forwarding and retracting movements of the fastening ring through the operation wire. The pair of clipping arms are configured to be respectively rotatable about an axis independently from the movement of the fastening ring by external force. At least one of the clipping arms can be further opened extendedly from the self-openable position by the external force.

### BRIEF DESCRIPTION OF THE DRAWINGS

FIG. 1 is a cross-sectional side view of a clipping instrument for an endoscope with arms thereof being extendedly opened according to an embodiment of the present invention.

FIG. 2 is a cross-sectional side view of the clipping instrument according to the embodiment of the present invention.

FIG. 3 is an exploded perspective view of a clip assembly of the clipping instrument according to the embodiment of the present invention.

FIG. 4 is a side view of the clip assembly of the clipping instrument in a ready position according to the embodiment of the present invention.

FIG. 5 is a side view of the clip assembly of the clipping instrument with arms in self-openable positions being opened by a fastening ring according to the embodiment of the present invention.

FIG. 6 is a side view of the clip assembly of the clipping instrument with the arms in extended positions being further extendedly opened by external force according to the embodiment of the present invention.

FIG. 7 is a side view of the clip assembly of the clipping instrument with the arms in closed positions being closed by the fastening ring according to the embodiment of the present invention.

FIG. 8 is an overall cross-sectional side view of the clipping instrument in operation according to the embodiment of the present invention.

FIG. 9 is an overall cross-sectional side view of the clipping instrument with the clip assembly clipping onto body tissue according to the embodiment of the present invention.

FIG. 10 is a side view of a conventional clipping instrument in operation.

### DETAILED DESCRIPTION OF THE EMBODIMENT

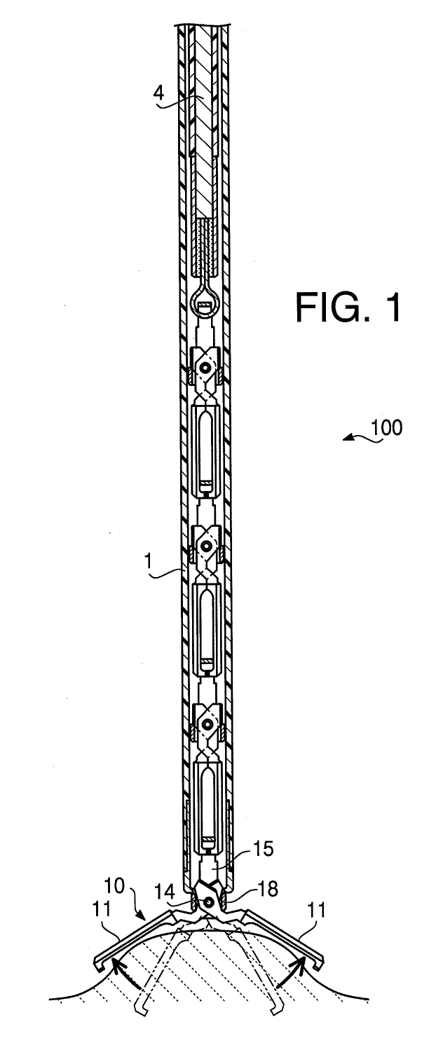

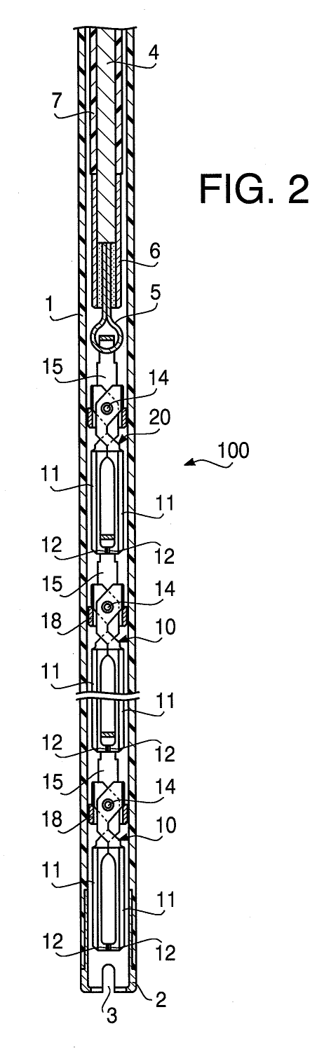

Hereinafter, referring to the accompanying drawings, an illustrative embodiment of the invention will be described. FIG. 1 is a cross-sectional side view of a clipping instrument 100 for an endoscope (not shown) with arms thereof being extendedly opened according to the embodiment of the present invention. The clipping instrument 100 includes a tubular flexible sheath 1. The flexible sheath 1 is made of, for example, poly-tetra-fluoro-ethylene and is inserted into a forceps channel (not shown) of the endoscope. FIG. 2 is a cross-sectional side view of the clipping instrument 100 with the arms tucked in a flexible sheath 1 according to the embodiment of the present invention.

The flexible sheath 1 is provided with a resilient sleeve 2, which is formed to be substantially cylindrical, fixed to a distal end of the flexible sheath 1, and is made of a metal. An inner diameter of the sleeve 2 is substantially equivalent to an inner diameter of the flexible sheath 1 and is formed to be substantially smaller at a distal end portion of the sleeve 2.

The sleeve 2 is further formed to have a plurality (for example, three or four) of slits 3, which are parallel to an axial direction thereof at the distal end portion. With these slits 3, the sleeve 2 can be deformed radially outwardly at the distal end portion.

Inside the flexible sheath 1, an operating wire 4 to be advanced and retracted in parallel with the axial direction of the flexible sheath 1 is provided. The operating wire 4 is connected to an operating unit (not shown) at a proximal end thereof and can be advanced and retracted in accordance with an operation given to the operating unit. The operating wire 4 is fixedly connected to a connecting loop 5 through a connecting pipe 6.

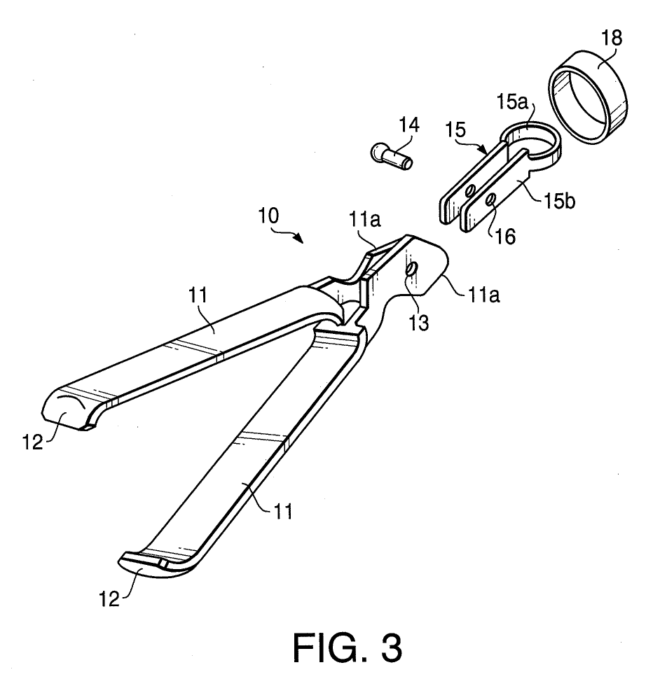

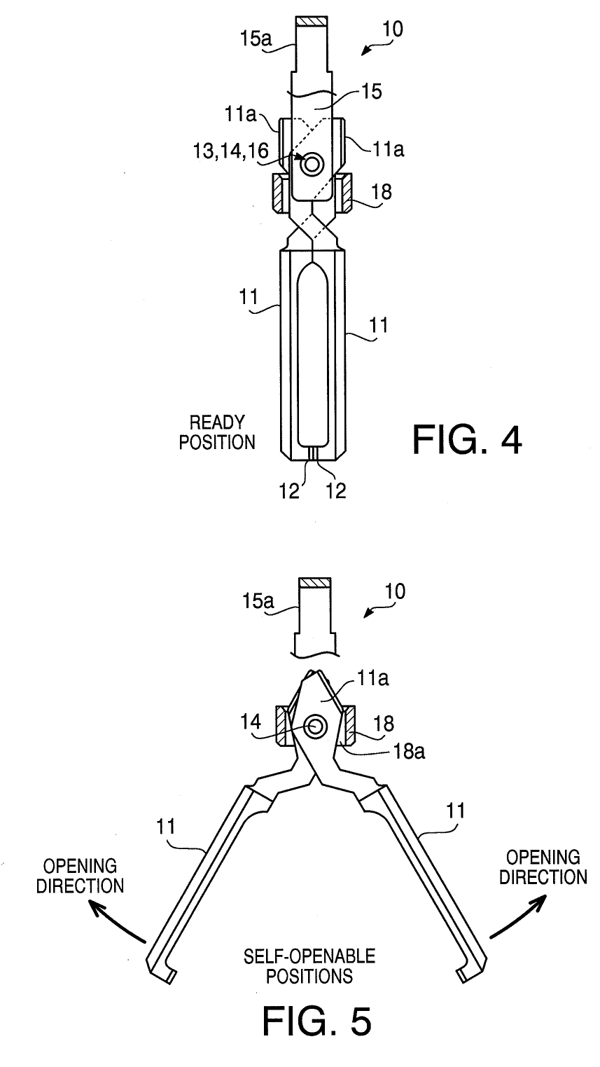

Further inside the flexible sheath 1, in vicinity to a distal end portion thereof, a plurality of clip assemblies 10 are aligned in line. FIG. 3 is an exploded perspective view of the clip assembly 10 of the clipping instrument 100 according to the embodiment of the present invention. FIG. 4 is a side view of the clip assembly 10 of the clipping instrument 100 in a ready position according to the embodiment of the present invention. The clip assembly 10 is provided with a pair of arms 11, each of which is individually formed and provided with a claw portion 12. The claw portion 12 is curved inward, when the arms 11 are assembled, so that the two claw portions 12 are opposed to each other to clutch a treatment object.

Each of the arms 11 is formed to have a through hole 13 in vicinity to a proximal end thereof. A shaft 14 supporting the arms 11 is penetrated through the through holes 13 of the arms 11 so that the arms 11, rotated about the shaft 14, can be arbitrarily opened and closed.

The clip assembly 10 is further provided with a tail portion 15, which includes an open-ringed connecting loop 15a at a proximal end thereof, a pair of plate portions 15b, which are parallel to each other and loosely sandwich the proximal end portion of the arms 11 therebetween, and through holes 16 through which the shaft 14 is penetrated. The tail portion 15 is connected to the operating wire 4 through another clip assembly 10 being aligned at a proximal side and a connecting clip 20, which will be described later.

Furthermore, the clip assembly 10 includes a fastening ring 18, by which the arms 11 are forcibly closed. When the arms 11 are allowed to be open, the fastening ring 18 is loosely fit around the proximal portion of the arms 11, as shown in FIG. 4.

FIG. 5 is a side view of the clip assembly 10 of the clipping instrument 100 with arms in self-openable positions being opened according to the embodiment of the present invention. When the fastening ring 18 is retracted rearward to an opening position shown in FIG. 5, in which the fastening ring 18 becomes in contact with cam portions 11 a of the arms 11. Thus, the arms 11 are opened.

When one of the clip assemblies 10 is projected out of the flexible sheath 1, the fastening ring 18 becomes in contact with the sleeve 2 at the distal portion thereof and is stopped thereat; therefore, the arms 11 can be opened. If the clip assembly 10 is forwarded further with more pressure, the fastening ring 18 outstretches the sleeve 2 and is projected forward from the sleeve 2.

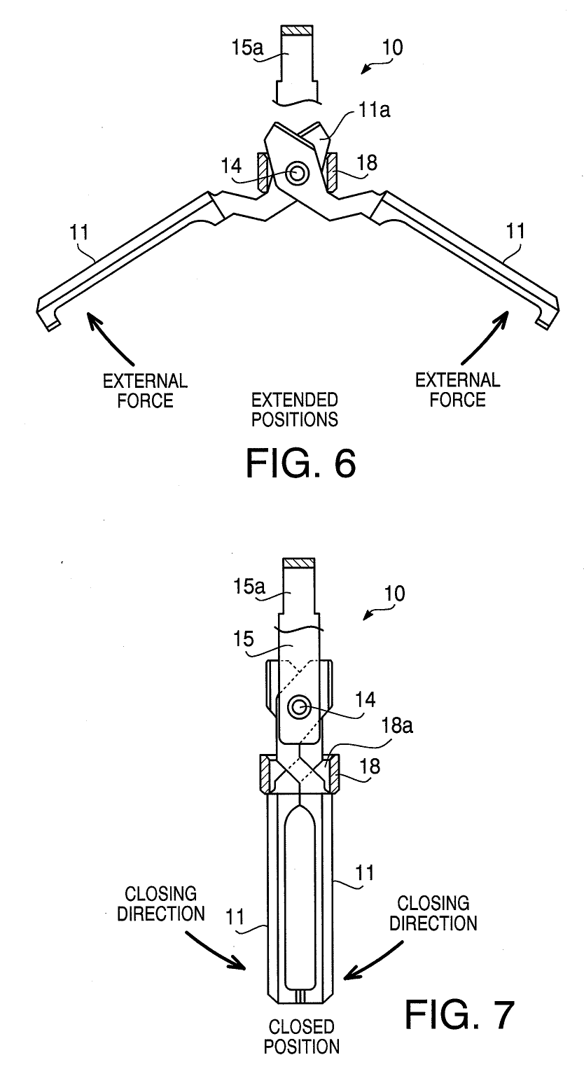

With the fastening ring 18 in the position to open the arms 11 fully, the arms 11 cannot be swung inward to the closing directions, as shown in FIG. 5. However, when the arms 11 are moved independently from the motion of the fastening ring 18, as shown in FIG. 6, the arms 11 can be respectively swung further in the opening directions to extended positions. Such movements are enabled by free space 18a provided inside the fastening ring 18 between an inner surface thereof and the cam portions 11a of the arms 11, which allows the cam portions 11a to further move in the opening directions. FIG. 6 is a side view of the clip assembly 10 of the clipping instrument 100 with the arms 11 in extended positions being opened further by external force according to the embodiment of the present invention. Thus, the arms 11 can be opened by external force further from self-openable positions, in which the arms 11 are opened to their extent by the fastening ring 18.

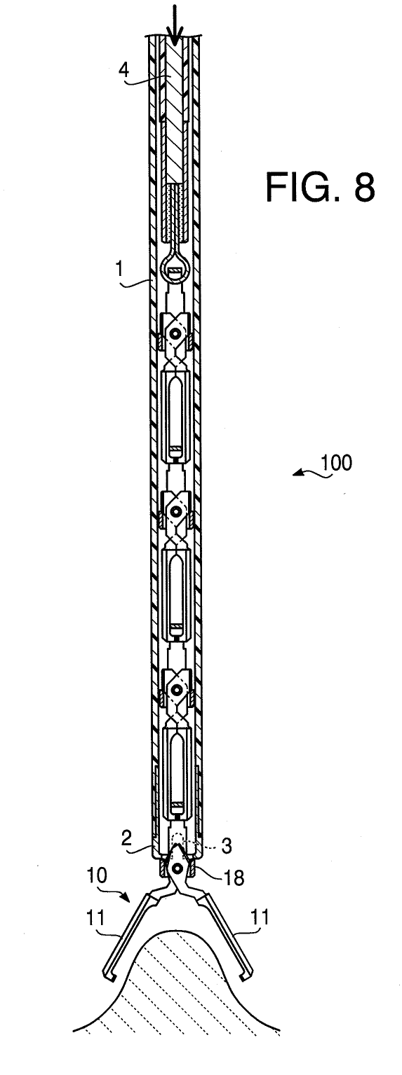

In reverse, when the fastening ring 18 is forwarded, as shown in FIG. 7, the arms 11 are forcibly closed. FIG. 7 is a side view of the clip assembly 10 of the clipping instrument 100 with arms 11 in closed positions being closed according to the embodiment of the present invention. When the tail portion 15 is retracted inward the flexible sheath 1 after the fastening ring 18 is once projected out of the sleeve 2, the fastening ring 18 becomes in contact with the sleeve 2 at the proximal portion thereof, and the arms 11 are closed.

Referring back to FIG. 2, in the flexible sheath 1 at the distal end portion, the clip assemblies 10 aligned in line are mutually connected. More specifically, the tail portion 15 of the clip assembly 10 in a position closer to the distal end of the flexible sheath 1 (front side) and the arms 1 1 of the clip assembly 10 in a position closer to the proximal end of the flexible sheath 1 (rear side) define a connected portion. The claw portions 12 of the arms 11 of the clip assembly 10 in the rear side are closed to clutch the connecting loop 15a of the clip assembly 10 positioned in the front side.

The clipping instrument 100 includes an undetachable clip assembly 20, which is undetachably connected to the connecting loop 5. Unlike the clip assemblies 10, the undetachable clip assembly 20 does not include the fastening ring 18; however, the remaining configuration of the detachable clip assembly 20 is identical to the clip assembly 10. The undetachable clip assembly 20 is connected to the tail portion 15 of the clip assembly 10 being located at the rear end of the aligned clip assemblies 10, i.e., the arms 11 of the undetachable clip assembly 20 are closed to clutch the tail portion 15 of the clip assembly 10 at the rear end.

The arms 11 of the plurality of mutually connected clip assemblies 10 and the undetachable clip assembly 20 are closed inside the flexible sheath 1 so that the clipping instrument 100 is inserted into the forceps channel of the endoscope.

FIG. 8 is an overall cross-sectional side view of the clipping instrument 100 in operation according to the embodiment of the present invention. As shown in FIG. 8, the clip assembly 10 at the distal end is projected outward from the flexible sheath 1 toward the body tissue to be clipped with the arms 11 opened to the self-openable positions by the fastening ring 18 so that the opened arms 11 can be pressed onto the body tissue.

When a size of a raised lump of the body tissue is larger than the extent of the opened arms 11 being opened by the fastening ring 18, the arms 11 can be further extendedly opened by external force provided from the body tissue, as shown in FIG. 1. Thus, the arms 11 can be pressed and clutched onto the body tissue without being damaged by the external force.

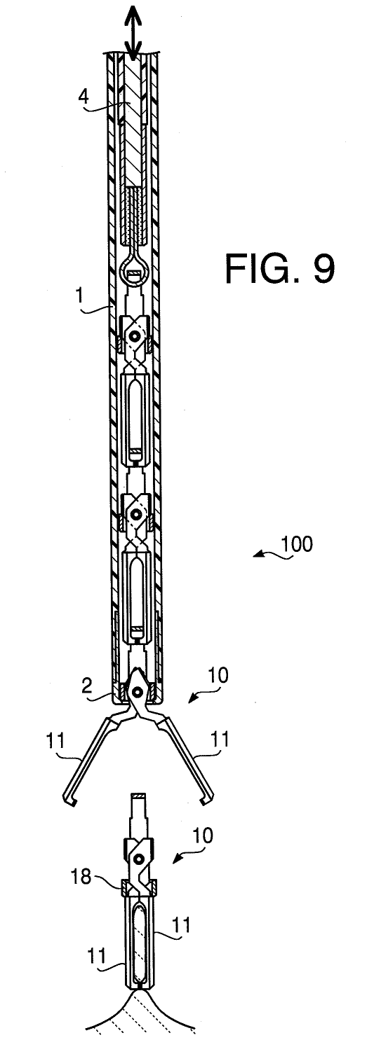

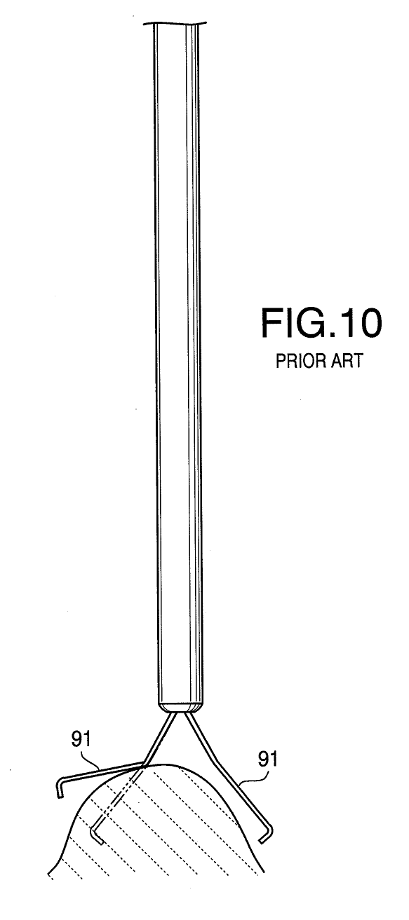

When the operation wire 4 is retracted toward the proximal end thereof, the arms 11 are forcibly closed by the fastening ring 18 to clip onto the body tissue. Further, when the clip assembly 10 arranged at the second from the front is projected from the flexible sheath 1, and the arms 11 of the second clip assembly 10 are opened, as shown in FIG. 9, the clip assembly 10 at the front end is detached from the second clip assembly 10 to indwell inside the body cavity.

Although an example of carrying out the invention has been described above, the present invention is not limited to the above described embodiments. For example, a single clip assembly 10 may be provided instead of two or more clip assemblies 10 in the clipping instrument 100.

The present disclosure relates to the subject matter contained in Japanese Patent Application No. P2007-153512, filed on Jun. 11, 2007, which is expressly incorporated herein by reference in its entirety.

What is claimed is:

1. A clipping instrument for an endoscope, comprising:
a flexible sheath to be inserted into an instrument channel of the endoscope;
an operation wire, which is configured to be forwarded and retracted in an axial direction of the flexible sheath; and
at least one detachable clip assembly, including a pair of openable and closable clipping arms and a fastening ring to encircle proximal portions of the clipping arms, arranged in vicinity of a distal end portion of the flexible sheath with the clipping arms closed, the clipping arms being configured to be projected forward and rotated in opening directions to open outside the flexible sheath to self-openable positions, in which the clipping arms are opened by an effect of the fastening ring to their self-openable extent, and to be rotated in closing directions to clip onto a treatment object outside the flexible sheath according to forwarding and retracting movements of the fastening ring through the operation wire,
wherein the pair of clipping arms are configured to be respectively rotatable about an axis independently from the movement of the fastening ring by external force; and
wherein at least one of the clipping arms can be further opened extendedly from the self-openable position by the external force.

2. The clipping instrument according to claim 1,
wherein the fastening ring is formed to have free space between an inner surface thereof and the encircled proximal portions of the clipping arms to allow the encircled proximal portions of the clipping arms to move in the opening directions so that the at least one of the clipping arms can be further opened extendedly from the self-openable position by the external force.

3. The clipping instrument according to claim 1, comprising
a plurality of the detachable clip assemblies aligned in line and an undetachable clip assembly, which is directly fixed to the operation wire and clipping onto one of the detachable clip assemblies, inside the flexible sheath.

4. The clipping instrument according to claim 1, wherein each of the proximal end portions of the clipping arms is formed to have a hole, through which a shaft to assemble the respectively rotatable clipping arms.

5. The clipping instrument according to claim 4,
wherein the detachable clip assembly includes a tail portion, which is formed independently from the clipping arms, rotatable about the axis with respect to the clipping arms, and connected with the clipping arms by the shaft.
